(19) United States
(12) Patent Application Publication
(10) Pub. No.: US 2015/0217142 A1
(43) Pub. Date: Aug. 6, 2015

(54) METHOD AND DEVICE FOR TREATMENT WITH COMBINATION ULTRASOUND-PHOTOTHERAPY TRANSDUCER

(71) Applicant: PHOTOSONIX MEDICAL, INC., Ambler, PA (US)

(72) Inventor: Mark E. Schafer, Lower Gwynedd, PA (US)

(21) Appl. No.: 14/685,803

(22) Filed: Apr. 14, 2015

Related U.S. Application Data
(63) Continuation-in-part of application No. 14/645,459, filed on Mar. 12, 2015.
Application No. 14/645,459 is a continuation of application No. 14/071,056, filed on Nov. 4, 2013, now Pat. No. 8,979,775.
Application No. 14/071,056 is a continuation of application No. 13/481,894, filed on May 28, 2012, now Pat. No. 8,574,174.
Application No. 13/481,894 is a division of application No. 12/189,829, filed on Aug. 12, 2008, now Pat. No. 8,206,326.
(60) Provisional application No. 61/033,470, filed on Mar. 4, 2008.

(51) Int. Cl. A61N 7/00; A61B 18/18; A61N 5/06

(52) U.S. Cl. CPC A61N 7/00; A61N 5/0613; A61N 5/0624; A61B 18/18; A61N 2005/0652; A61N 2005/0629

(57) ABSTRACT

A treatment device is provided that enables a volume of tissue located beneath the device to simultaneously receive ultrasound and light stimulation. According to one embodiment, the device includes an ultrasound transducer, a light source, and a faceplate extending across a face of the transducer for providing a tissue contacting and ultrasound energy coupling surface of the device. The faceplate is transparent or translucent to the light generated by the light source, and the faceplate transfers the ultrasound and light energy into the tissue or into an element which transmits these energies into the tissue. A controller operates the ultrasound transducer and light source in a pulsed manner to produce discrete pulses of energy, so as to provide higher amplitude energy treatments without negatively impacting heat generation of the device or the benefits of co-locating both types of energy treatment.

### CROSS-REFERENCE TO RELATED APPLICATIONS

This application is a continuation-in-part application of U.S. patent application Ser. No. 14/645,459, filed Mar. 12, 2015 (pending), which is a continuation application of U.S. patent application Ser. No. 14/071,056, filed Nov. 4, 2013, now U.S. Pat. No. 8,979,775, which is a continuation application of U.S. patent application Ser. No. 13/481,894, filed May 28, 2012, now U.S. Pat. No. 8,574,174, which is a divisional application of U.S. patent application Ser. No. 12/189,829, filed Aug. 12, 2008, now U.S. Pat. No. 8,206,326, which claims the benefit of U.S. Provisional Application No. 61/033,470, filed Mar. 4, 2008 (expired). The disclosure of each of these applications is hereby incorporated by reference herein in its entirety for all purposes.

### BACKGROUND

The present invention relates to ultrasonics, more specifically, to the application of ultrasound energy for physical therapy and wound or tissue treatment.

Ultrasound energy has been used for over 50 years as a therapeutic treatment modality in physical therapy and sports medicine. Recently, lower intensity ultrasound has been used to accelerate the healing of bone fractures and both fresh and long term non-healing wounds. For purpose of example, U.S. Pat. No. 7,282,036 issued to Masuda; U.S. Pat. No. 7,195,603 issued to Yamazaki et al.; U.S. Pat. No. 7,022,089 issued to Ooba et al.; U.S. Pat. No. 6,273,864 issued to Duarte et al.; U.S. Pat. No. 5,690,608 issued to Watanabe et al.; U.S. Pat. No. 5,549,544 issued to Young et al.; U.S. Pat. No. 5,520,612 issued to Winder et al.; U.S. Pat. No. 4,530,360 issued to Duarte; U.S. Pat. No. 3,2828,769 issued to Mettler; U.S. Pat. No. 2,830,578 issued to De Groff; and U.S. Pat. No. 2,283,285 issued to Pohlman disclose various therapy apparatus including handpieces containing ultrasound transducers.

It has also been shown that phototherapy, that is, the application of light at different wavelengths, can have a therapeutic effect on skin and underlying tissue. For example, see U.S. Pat. No. 6,443,978 issued to Zharov; U.S. Pat. No. 6,290,713 issued to Russell; U.S. Pat. No. 5,913,883 issued to Alexander et al.; U.S. Pat. No. 5,358,503 issued to Bertwell et al.; U.S. Pat. No. 5,259,380 issued to Mendes et al.; and U.S. Pat. No. 4,930,504 issued to Diamantopoulos et al. for various phototherapy treatment apparatus.

Accordingly, medical and cosmetic devices have been built and sold which provide for either treatment modality. With respect to ultrasound devices, it is conventional to position the ultrasound transducer behind an opaque, metal front face of the device that directly contacts the skin of the patient. The conventional faceplate and ultrasound transducer covers the tissue region being treated so that light cannot be readily applied to the same area of skin. Thus, the presence of the front face provides an obstacle to the simultaneous application of ultrasound and light to tissue underlying the front face.

Some therapeutic ultrasound systems include separate ultrasound and light sources (e.g. Hill Therapeutics HF54, BTL Physio BTL-4800SL), so that the operator can conveniently switch between the two treatments. Also see U.S. Pat. No. 7,090,649 issued to Kang which discloses a skin management system that includes various separate handpieces capable of performing different functions. Some of the handpieces include light sources and others include an ultrasound transducer. However, it is not possible to treat with both modalities at the same time with these devices, let alone at the same time on the same volume of tissue.

Some prior art systems have used smaller transducers, or “sound heads,” such that the separate light source can be applied nearby. However, again, these do not allow for simultaneous stimulation by both ultrasound and light over the same tissue region.

Other systems have included a small light aperture in the middle of the transducer, but for these units, the light source is used not for therapy, but as an adjunct to the diagnostic use of the ultrasound. For example, transducers designed for the purpose of measuring the size of the human eye, called A-scan devices, may have a light emitting from their center, so that the patient can be told to look at the light as the probe is placed on the eye (Paradigm Medical P-2000). In this way, the “fixation light” causes the patient to properly hold their gaze, thus allowing the transducer to make proper contact with the corneal surface. This approach is not considered for any therapeutic application of either the ultrasound or the light. Another similar example is provided by U.S. Pat. No. 6,702,749 issued to Paladini et al. which discloses an optical needle guide for an ultrasound guided needle biopsy.

U.S. Pat. Nos. 6,533,803 and 6,761,729 issued to Babaev disclose a handpiece of a treatment device used to deliver a spray of liquid drugs and laser energy to a wound. In use, the handpiece is positioned a spaced distance (about 0.5 inches) from the wound and is activated to simultaneously apply a spray of atomized particles of a drug delivered via ultrasonic waves and a beam of light to the wound.

U.S. Pat. No. 5,699,804 issued to Rattner discloses an apparatus having an X-ray transparent ultrasound transducer provided as a piezoelectrically activated polymer (PVDF) foil, and U.S. Pat. No. 6,361,509 issued to Reuner discloses a similar device that includes sources for generating acoustic and light waves. The light source emits a focused beam of visible light along the acoustic wave axis for the purpose of making the acoustic axis visible.

U.S. Patent Application Publication No. 2004/0236252 of Muzzi et al. discloses apparatus for treating cellulitus. The apparatus includes a handpiece having a face plate adapted to contact the skin of a patient and provide mechanical message via pulsed suction through a central open channel that extends through the face plate. Simultaneous with the pulsed suction, biostimulation is provided via six laser diodes distributed uniformly on the face plate around the channel opening.

Finally, U.S. Pat. No. 7,273,459 issued to Desilets et al.; U.S. Pat. No. 5,316,000 issued to Chapelon et al.; U.S. Pat. No. 4,683,396 issued to Takeuchi et al.; U.S. Pat. No. 4,658,176 issued to Nakaya et al.; and U.S. Pat. No. 4,412,148 issued to Klicker et al. disclose composite piezoelectric transducers in general.

### SUMMARY

The present invention combines ultrasound treatment of tissue with light treatment of tissue to provide one of several desirable treatment effects on the tissue. In one particular treatment device, the light energy from the light source is allowed to pass through the ultrasound transducer and into the tissue, while the ultrasound transducer applies ultrasound energy at the same location of the tissue. To this end, the light and ultrasound treatments are co-located on the tissue. Furthermore, the treatment method performed includes pulsing the energy application of one or both of the light and ultrasound to reduce the overall energy application and temperature increase experienced in the tissue being treated. This pulsed application of light and ultrasound enables higher energy amplitudes to be applied for deeper and/or more thorough tissue treatments, while also varying the energy application frequently enough to avoid allowing the tissue being treated to adjust for the energy and reduce the effectiveness thereof. Furthermore, because the complementary effects provided by the ultrasound energy for enhancing the light energy treatment last for a small period of time after the ultrasound treatment stops, the pulsed treatment with rapid pulses results in effectively the same result on the tissue as continuous treatments, and perhaps even better treatment effectiveness for less overall energy use.

### BRIEF DESCRIPTION OF THE DRAWINGS

The foregoing and other objects, features and advantages of the present invention should become apparent from the following description when taken in conjunction with the accompanying drawings, in which:

FIGS. 1A-1C are cross-sectional and bottom plan views of the construction of an ultrasound soundhead according to the prior art;

FIGS. 2A-2C are cross-sectional and top plan views of the structure of piezocomposite transducers according to the present invention;

FIG. 3 is a top plan view of means to provide electrical connections to the piezocomposite transducer of the present invention;

FIGS. 4A-4C are cross-sectional and top plan views of an alternate structure of the piezocomposite transducer according to the present invention;

FIG. 5 is a top plan view of a means to provide electrical connections to the alternate piezocomposite transducer structure of the present invention;

FIG. 6 is a cross sectional view of the overall structure of the treatment device according to the present invention, showing both the ultrasound transducer structure and light sources located behind the transducer structure;

FIGS. 7A-7C are a top plan view, a bottom plan view, and a cross sectional view of an alternate embodiment of the present invention, one which provides for an annular array of sub-elements;

FIG. 8 is a top plan view of an alternate embodiment having an annular array of sub-elements with a selected amount of ultrasound elements removed in order to allow more light to pass through the transducer;

FIGS. 9A and 9B are a cross-sectional and plan view of an alternative embodiment according to the present invention which includes a light source that is disposed around the rim of an ultrasound transducer;

FIG. 10 is a perspective view of an alternative embodiment of the present invention that uses a ring transducer element to excite the front face layer, and allows maximum light transmission; and

FIGS. 11A and 11B are top plan views of alternative embodiments having one or more ring transducer elements;

FIG. 12 is a graphical representation of a continuous waveform of energy emitted for treating bacteria according to an embodiment of the invention;

FIG. 13 is a simplified graphical representation of a pulsed waveform of energy emitted for treating bacteria according to an embodiment of the invention;

FIG. 14 is a simplified graphical representation of pulsed ultrasound and light emitted in a temporally alternating and interleaved manner for treating bacteria according to an embodiment of the invention;

FIG. 15 is a simplified graphical representation of pulsed ultrasound and light emitted in a temporally overlapping manner for treating bacteria according to an embodiment of the invention;

FIG. 16 is an image of a bacterial biofilm treated at one region with light only and at another region with combined light and ultrasound to show experimental test results in accordance with the embodiments of the invention;

FIG. 17 is a table showing experimental parameters and test results corresponding to a treatment of bacteria with combined ultrasound and light energies according to another embodiment of the invention; and

FIG. 18 is a schematic view showing an experimental setup, including a photo-acoustic treatment device, according to another embodiment of the invention, this experimental setup being the treatment device used to produce the results shown in FIG. 17.

### DETAILED DESCRIPTION

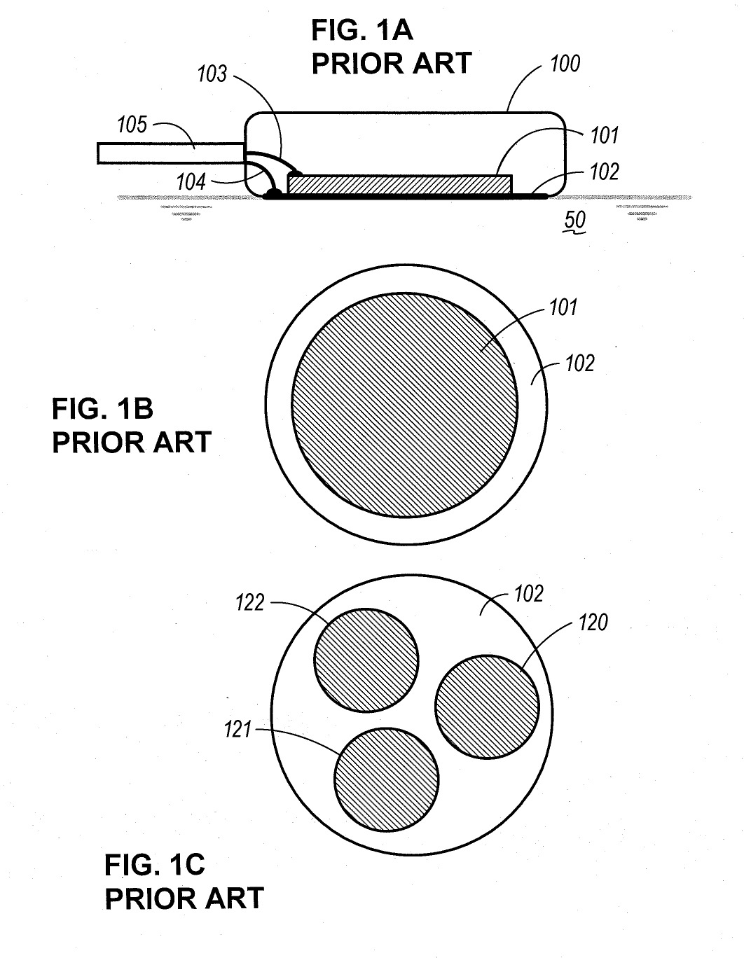

FIGS. 1A-1C depict a soundhead of design typical of the prior art. FIG. 1A is a simplified cutaway diagram of the soundhead in contact with tissue 50. The soundhead consists of housing 100, which contains the ultrasound transducer element 101, attached to the faceplate 102 which makes contact with tissue 50. The faceplate 102 is typically metal, either stainless steel or aluminum. Cable 105 attaches to housing 100. Lead 103 of cable 105 attaches to the rear of transducer element 101, and lead 104 of cable 105 attaches to the front of transducer element 101, typically through an electrical connection to the metal faceplate 102. Other means of connection have been used, including “wrap around” electrodes on transducer element 101 such that the electrical connection to the front of the transducer element is brought around to an isolated portion of the rear of the transducer element. The exact means are not necessarily important to the present invention.

The current art in ultrasound therapy uses soundheads operating at frequencies between 1 and 5 MHz, with 1 and 3 MHz being the most popular. The size of the soundhead ranges from 20 mm to 50 mm in diameter, depending upon the region of the body to be treated.

Transducer element 101 is typically a solid, continuous piece of piezoceramic material, as can be purchased from a number of vendors (e.g. Morgan Electro Ceramics, Bedford, Ohio). Most typically it is furnished as a circular disk, as can be seen in FIG. 1B. This approach is relatively low cost and robust.

FIG. 1C depicts another embodiment of the prior art, in which the single transducer element 101 attached to faceplate 102 is replaced by three smaller elements 120, 121, and 122, all still attached to faceplate 102. This design is sometimes employed to modify the structure of the ultrasonic beam emitted into tissue 50. The modification may be desirable in some instances to control the unwanted focusing of the ultrasound energy.

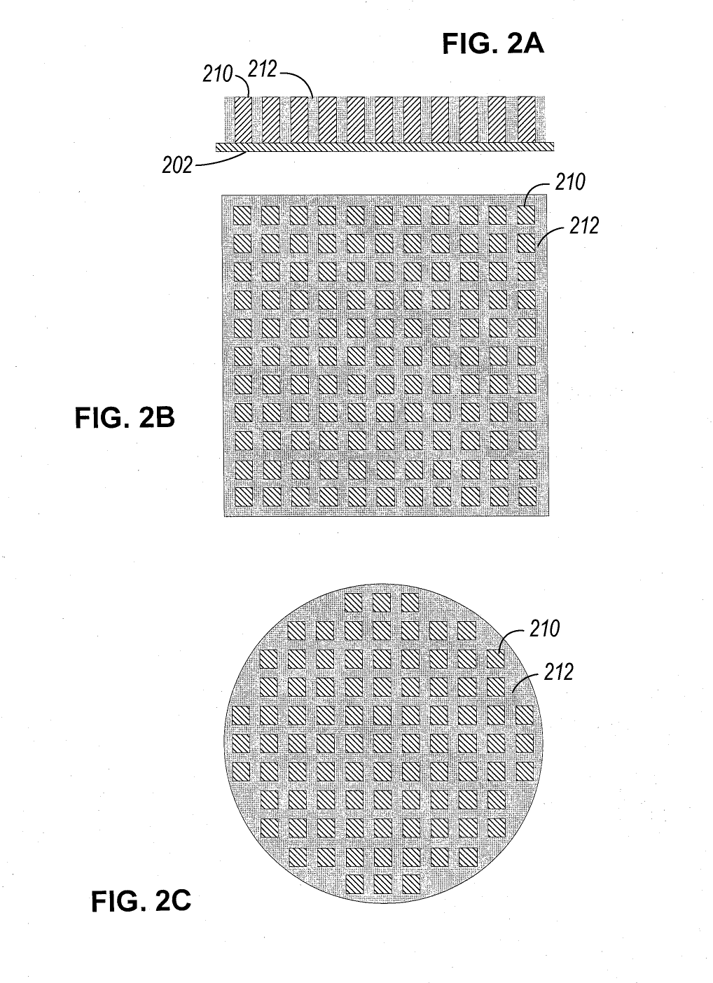

FIGS. 2A-2C depict transducer structures according to the present invention and are comprised not of a single continuous element, but of a plurality of smaller individual elements 210. This approach is known as a piezocomposite, because it is a composite of individual elements 210, imbedded or surrounded by a polymer matrix material 212. This approach has been known for a number of decades. See, for example: R. E. Newnham et al., “Composite Piezoelectric Transducers,” Materials in Engineering, vol. 2, December 1980, pp. 93-106; T. R. Gururaja et al., “Piezoelectric Ceramic-Polymer Composites For Transducer Applications,” Electronic Ceramics, 1987, pp. 92-145; H. P. Savakus et al., “PZT-Epoxy Piezoelectric Transducers,” Materials Research Bulletin, vol. 16, 1981, pp. 677-680; U.S. Pat. No. 4,412,148 issued to Klicker et al.; U.S. Pat. No. 4,658,176 issued to Nakaya et al.; and U.S. Pat. No. 4,683,396 issued to Takeuchi et al. The structure has the advantage of improved performance at lower weight when compared to a solid ceramic transducer at the same frequency and size. This approach is known in the art as a “1-3” composite, because the transducer ceramic material exhibits connectivity only along one axis (giving rise to the “1”), while the polymer matrix material exhibits connectivity along all three axial directions (giving rise to the “3”). The ceramic connectivity is always listed first, followed by the polymer matrix connectivity.

FIG. 2A is a schematic cutaway view of a piezocomposite transducer, with elements 210 imbedded in a matrix material 212, attached to a faceplate 202. FIG. 2B shows a schematic top view of said piezocomposite transducer, showing the relative placement of the individual sub-elements 210. FIG. 2C depicts the application of this approach to a circular aperture configuration.

To date, this type of transducer is commonly found in diagnostic ultrasound equipment, where the increased cost of manufacture is balanced by the much improved performance afforded by the design. In designing these transducers for said diagnostic or therapeutic purposes, the choice of the polymer matrix material 212 and the front face material 202 are determined solely by their ultrasonic properties, and not their light transmission properties.

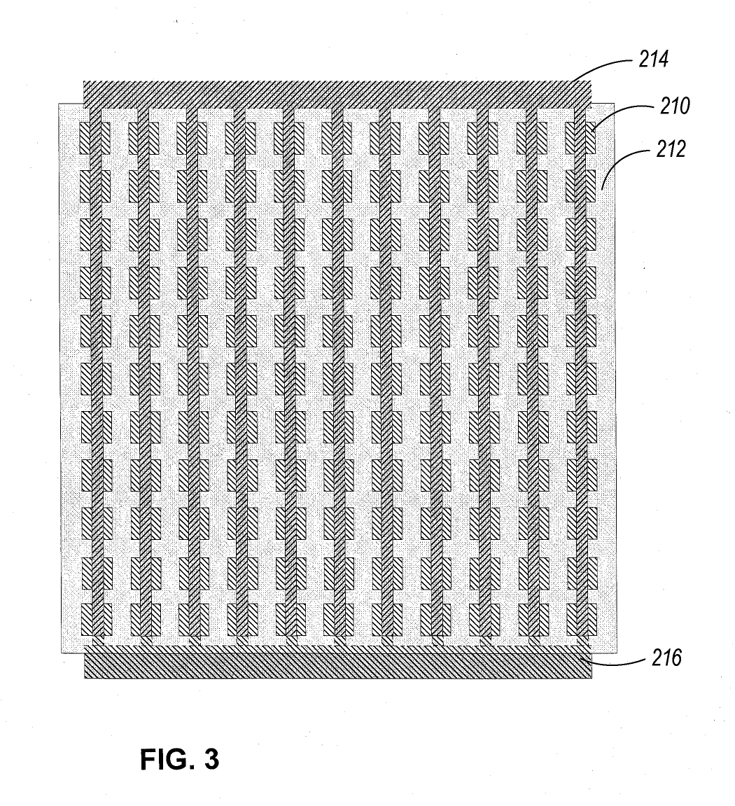

FIG. 3 depicts a novel means of the present invention for establishing electrical contact with the individual elements 210 of the 1-3 composite material. The electrodes on the top 214 and bottom 216 may be applied by a silk screening, electro-etching, or plasma sputtering operation. In most diagnostic transducers where light transmission is not a design requirement, these electrodes are typically applied in a plating operation and cover the entirety of the top and bottom surfaces of the transducer. The electrode material may be silver, copper, gold, or other highly conductive material, depending upon the method used for its application. It is important to note the specific design goal of the present invention is to provide for light transmission through the ultrasound transducer structure, so it is important that the electrode material have gaps so as to allow light to pass through the electrodes.

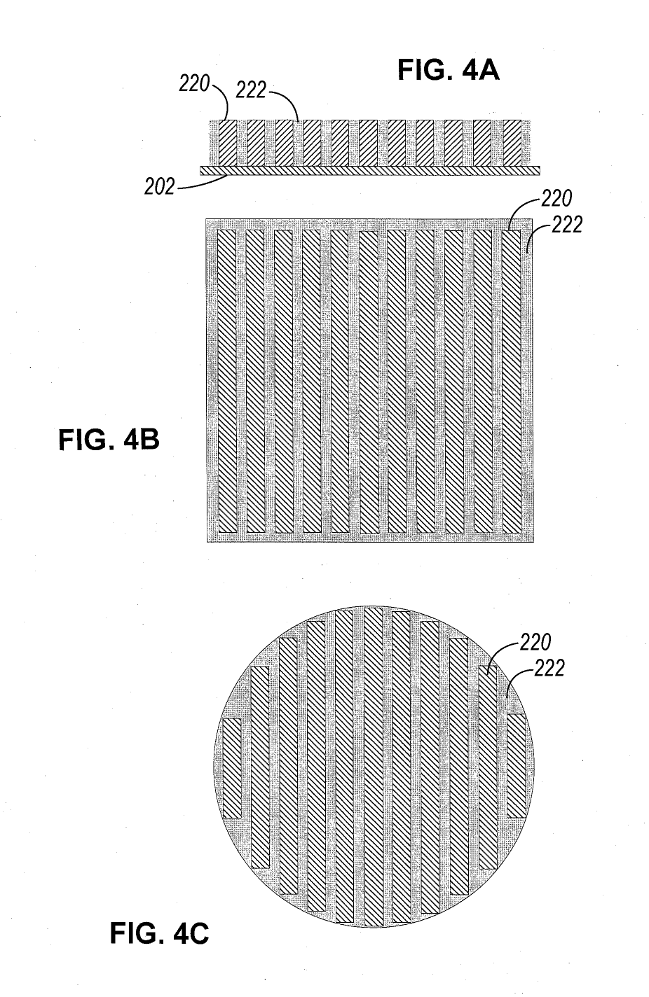

FIGS. 4A-4C depict an alternate embodiment of the present invention which uses a “2-2” connectivity of the transducer ceramic and polymer matrix. The individual elements 220 imbedded in a polymer matrix material 222 are now in the form of bar structures rather than post structures. This approach results in a higher overall volume fraction of ceramic, which leads to a higher acoustic output on transmit. Again, as can be seen in FIGS. 4B and 4C, the overall shape of the transducer may be rectangular, circular or other shapes as the situation demands.

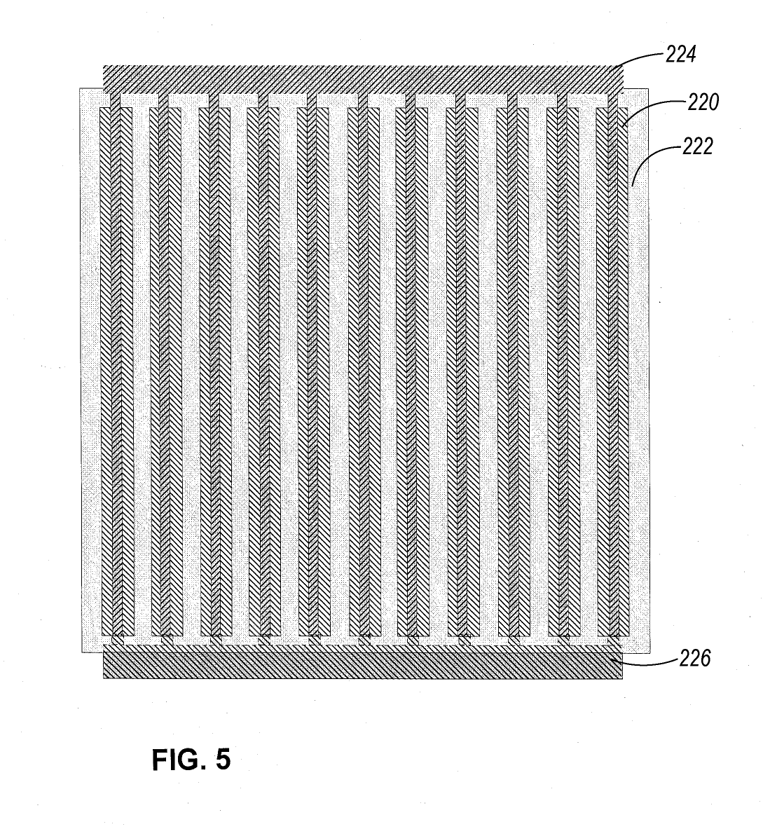

FIG. 5 depicts a means for establishing electrical contact with the individual elements 220 of the 2-2 composite material. Electrode 224 is on the top of the structure and electrode 226 is on the bottom, and may be applied by the methods enumerated above. Note that the 2-2 design is more efficient in the balance of light transmission and transducer area, in that with the 1-3 design, the area between some elements may be blocked by the presence of the electrode material. In the 2-2 design, the electrode and ceramic are co-extant.

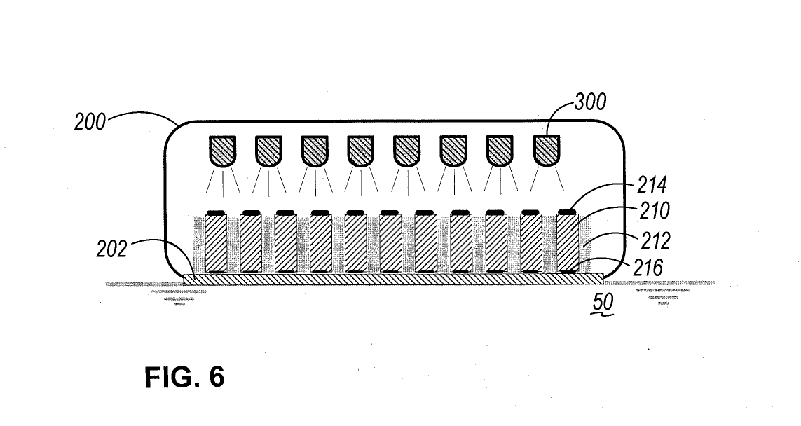

FIG. 6 depicts in schematic form the overall structure of the treatment device of the present invention, with transducer elements 210 imbedded in polymer 212, connected by electrodes 214 on the top and 216 on the bottom, bound to faceplate 202 in contact with tissue 50, contained within housing 200, and transilluminated by a plurality of light sources 300. By choosing the polymer matrix material 212 to have both acceptable acoustic properties and good light transmission properties (e.g. Epotek 310, Epoxy Technology, Billerica Mass.), it is possible to have the tissue 50 beneath the soundhead receive simultaneous ultrasound and light stimulation.

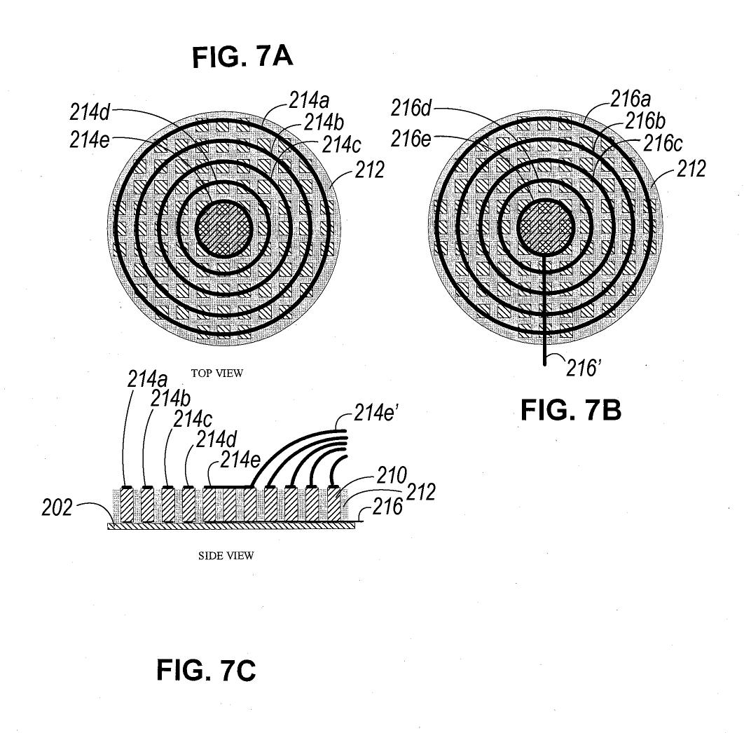

FIGS. 7A-7C depict in schematic form alternate embodiments of the 1-3 composite design, demonstrating alternatives which may be implemented using the methods taught by the present invention. In FIG. 7A, electrodes 214a, 214b, 214c, 214d, and 214e create concentric ring patterns of elements 210. By changing the phase of the electrical drive signals to each ring, it is possible to focus or defocus the resultant ultrasound distribution pattern within the tissue. There may be cases in which the ultrasound is required to be focused in order to achieve a desired minimum ultrasound excitation level within a small volume of tissue. Or it may be desirable to insonify a larger volume of tissue at a lower ultrasound exposure level. Note that electrode 214e also comprises a circular area, which may limit the overall light transmission. FIG. 7B depicts the connections to the bottom side of the transducer element. Here all electrodes 216a-216e are connected to a common electrode 216′.

FIG. 7C shows a side view of the connection scheme, with each ring electrode on the top portion connected via a lead or wire from the rear of the transducer, e.g 214e′. The cable to this transducer would consist of multiple connections which would allow the individual rings to be phased as noted.

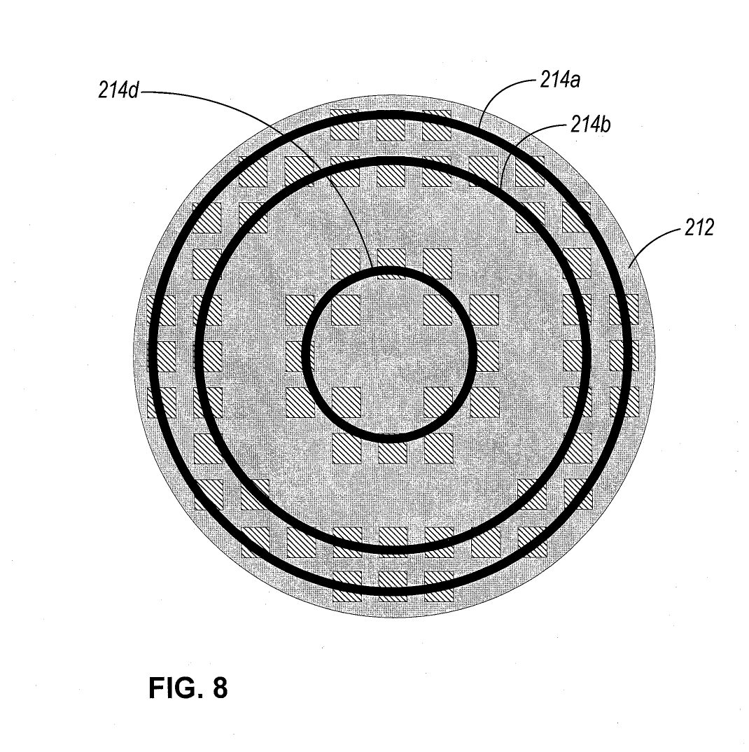

FIG. 8 depicts an example in which selected ultrasound elements have been removed from that shown in FIG. 7A in order to allow more light to pass through the transducer. The elements can be removed in the processing stage before the addition of the polymer/epoxy matrix material. Electrodes 214a, 214c, and 214d may all be connected together electrically, or may be energized separately as noted above. In this case, electrode 214d is only a circular ring, and not an entire disk. The individual rings may be connected as previously depicted in FIGS. 7A-7C.

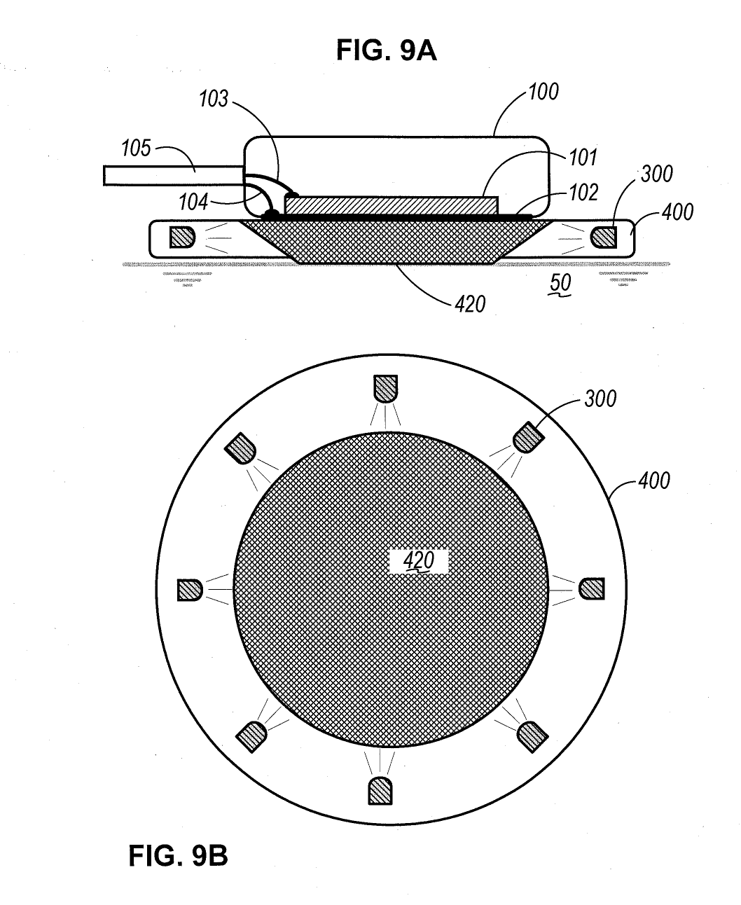

FIGS. 9A and 9B depict an alternative approach which also permits at least some overlap of the ultrasound beam and a light from a light source disposed around the rim of the ultrasound transducer. This approach would be suitable as a retrofit to an existing ultrasound transducer of the prior art. An opto-acoustic material 420 is interposed between the faceplate 102 of a soundhead of the prior art and the tissue surface 50. Disposed around the periphery of said opto-acoustic material is a plurality of light sources 300 within a housing 400. The opto-acoustic material 420 provides acoustic coupling for the ultrasound energy from the transducer 101. It also reflects the light energy from the light sources 300 downward towards the tissue. This may be done by simple diffusion, e.g. by using a translucent material, or diffuse reflection, e.g. introducing light reflecting particles such as gold or silver particles within the material. This may be done if the material 420 is formed as a cast material, for instance as a mixture of fine gold particles dispersed within a cast epoxy.

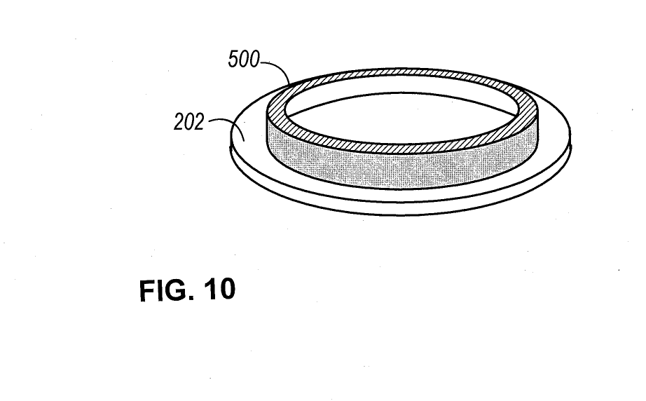

FIG. 10 depicts an alternative embodiment wherein the balance of light and ultrasound output emphasizes light output. The transducer element is in the form of a ring 500. Such transducer elements are known to those skilled in the art and may be obtained from a number of suppliers (e.g. Morgan Matroc, Ohio). Transducer elements in this form have been used to drive, for instance, ultrasonic surgical equipment. The thickness of the element sets the resonant frequency of the device. One advantage of this approach is that the entire center is available for light transmission through the front face 202. A device built on this principle can be structured similarly to that shown in FIG. 6, with illumination elements 300 positioned directly behind front face 202. This approach has the advantage of structural simplicity, and potentially high light output. However, the ultrasound output which may be obtained with this approach is not as high as that obtained with the other transducer configurations disclosed, nor is the ultrasound field as uniform in the region directly in the center of the transducer. The front face 202 may be considered as a radiating face, like a drum head, depending upon frequency. The higher the frequency, however, the more the ring element 500 will produce an annular region of ultrasound exposure, with less intensity in the center region.

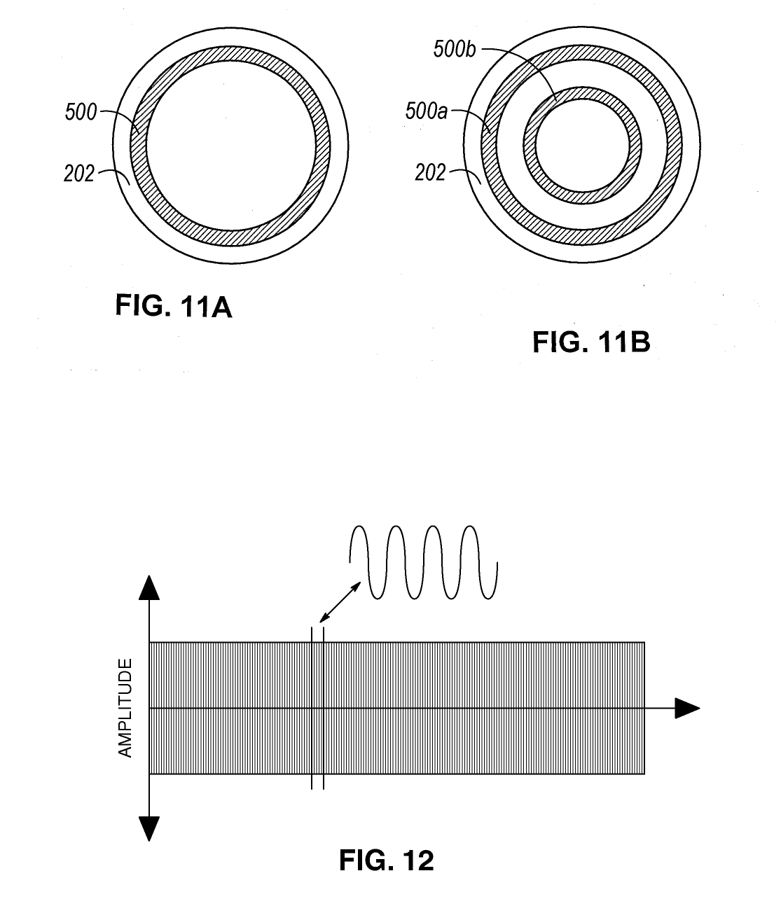

FIGS. 11A and 11B depict the above mentioned alternative embodiment and include one or a plurality of ring transducer elements. FIG. 11B shows only two elements, 500a and 500b, but those skilled in the art would appreciate that additional elements are possible. These elements could be wired in a manner similar to that shown in FIGS. 7A-7C and 8.

The primary functional difference between the present invention and the prior art is the capability to have light pass through the transducer element, allowing for simultaneous ultrasound and light stimulation of a tissue volume. The structural difference is the use of piezocomposite transducer technology, with the additional design constraints of 1) using a polymer or epoxy matrix material which is transparent or sufficiently translucent; 2) using a transparent or sufficiently translucent front plate rather than a solid metal plate; 3) using a specific pattern of electrodes which provide connection to the individual ultrasound elements within the piezocomposite but also minimize the optical shadowing of the light sources.

Although as noted, there are ultrasound transducers with a small aperture in the center for a “fixation” light, this type of transducer is not designed for a combination therapy. The fixation light is used through a small hole, on the order of several millimeters in diameter. This hole is drilled through the transducer assembly. Other transducers have been designed with holes in or around the transducer face: for instance, transducers used with biopsy needles have holes or slots in them to accommodate the needle. All these transducers are designed for diagnostic purposes and are not intended to have a therapeutic use. Those persons familiar with these devices, e.g. those involved in ophthalmic ultrasound, are not generally aware of the potential need to apply phototherapy and ultrasound therapy to the same tissue volume simultaneously.

Another issue is the fundamental design of the ultrasound transducer. Those skilled in the art of therapeutic transducers have long used metal front face transducers in order to provide: 1) electrical shielding and grounding for patient safety (although some systems have been described which apply an electrical current through the soundhead to provide ancillary electrical stimulation therapy); 2) heat sinking capacity to reduce any unwanted heat generated by the transducer element within the soundhead; and 3) to provide some mechanical stability to the solid ceramic transducer when it is operated at a high drive level. In many current ultrasound therapy devices, the goal is to create deep heating within the tissue bed. Thus the ultrasound powers are relatively high, especially in comparison to diagnostic ultrasound power output levels. To obtain these levels, the metal front face is recommended for the reasons given above, especially with regard to heat capacity of the soundhead and the mechanical stability requirements.

Further, piezocomposites have not been extensively used for high ultrasound output. Because while the design offers certain advantages, especially with regard to the bandwidth of the transducer and coupling of the ultrasound energy, the mechanical losses inherent in the polymer/epoxy matrix absorb too much of the available energy, reducing their efficiency at high drive levels. This inefficiency causes heating within the transducer, which can degrade the polymer/epoxy matrix material.

However, it has been shown that beneficial tissue response can occur at much lower levels of ultrasound stimulation. For example, see U.S. Pat. No. 4,530,360 issued to Duarte and U.S. Pat. No. 5,520,612 issued to Winder et al., where the level of ultrasound intensity is less than 100 mW/cm² and on the order of 30 mW/cm². This obviates the need for high transducer drive levels, and thus it is not necessary to use a metal front face. Further, these lower ultrasound levels are compatible with piezocomposite transducer designs.

However, the most critical insight is the intimate knowledge of the design of piezocomposite transducers. Knowing the pattern of ceramic elements imbedded in a polymer/epoxy matrix allowed for the design concept of using a transparent or translucent matrix material. In practice, a translucent material, especially for the front face, would better diffuse the treatment light energy, allowing for more uniform coverage. The trade off is in the amount of total light to reach the skin or tissue surface.

Localized treatment using light sources (also known as topical photodynamic therapy, PDT) has developed into an established modality for a number of conditions, including non-melanoma skin cancers, acne and photorejuvenation. These therapeutic devices use low level lasers (LLLT) or bright light emitting diodes (LEDs) of different colors (wavelengths). Those skilled in the art of such devices are generally not aware of the capabilities of ultrasound for tissue treatment, and are specifically not aware of the possibility of modifications to the ultrasound transducer as have been described herein.

As noted above, the primary advantage of the disclosed invention is the ability to treat tissue with both ultrasound and light at the same time, at the same location. This affords new treatment possibilities, including the synergistic treatment of different diseases. As an example, both ultrasound and light have been demonstrated to have a positive effect on wound repair. Although the complete mechanisms of repair are not completely known, there are indications that ultrasound and light affect different tissue structures, which would lead to the possibility of complementary effects when the two modalities are combined.

Another possibility is the use of ultrasound to change the permeability of skin or other tissue. This would allow topical application of drugs to more efficiently travel to their target tissues. As an example, 5-aminolevulinic acid (5-ALA) is used topically in the treatment of non-melanoma skin cancers. 5-ALA is converted inside the cell into protoporphyrin IX (PPIX), a natural photosensitiser that is usually present in too small a quantity to cause photosensitivity. When 5-ALA is applied to the surface of skin tumors it is preferentially absorbed and converted by dysplastic/neoplastic cells. When these cells are treated with light therapy, the resulting photodynamic reaction produces reactive oxygen species, leading to cell death. Ultrasound could accelerate the uptake process by increasing the permeability of the cell walls.

Another advantage of the invention disclosed herein is the use of a non-metallic front face material. While the metal front face does have some advantages for high power ultrasound applications, it is actually a very poor choice as a coupling material between the transducer and the skin. This is because the acoustic impedance of the metal is higher than the ceramic material, which causes an impedance mismatch. This causes ultrasound energy to be reflected back into the transducer element, lowering overall efficiency. Ideally, the front face should be an acoustic matching layer, such that its acoustic impedance lies between that of the transducer and the tissue. The principles of acoustic matching layers are well known (cf. F. Kremkau, “Diagnostic Ultrasound: Principles and Instruments,” 6th Ed. ISBN 0-7216-9330-X, 2002, pp. 66). Plastic materials such as Lucite® are both transparent and acoustically suitable for use as a front face material. There are design tradeoffs with the front face in terms of the light attenuation as a function of light wavelength, accelerated aging of materials due to light exposure, etc. which will be obvious to those skilled in the art of plastics usage. To these requirements is the additional design requirement, as noted, of acoustic matching. Again, those skilled in the art of transducer design are aware of the needs for the proper material, thickness, bonding, etc.

Another advantage of the invention herein disclosed is the capability of shaping the front of the transducer into a non-planar form. Because the piezocomposite is made from individual elements, especially in the case of the 1-3 design, it is possible to shape the overall structure into a spherical cap, either concave (which is often done in diagnostic imaging to impart focusing properties) or convex (which would allow for the dispersion of the ultrasound over a wider area). It would also allow the photo-ultrasound transducer to be shaped into novel configurations, such as into a cylindrical shape. Such a shape could be used, for instance, for treatment within body cavities.

Another advantage of the design is to further combine the utility of the ultrasound with a light source and use the ultrasound transducer not only as a treatment device but also as a diagnostic device. Specifically, the ultrasound transducer could be used for imaging a portion of the tissue. Moreover, the ultrasound transducer could be employed using Doppler principles (cf. F. Kremkau, Chapter 5) to monitor blood flow in the tissue. This could be employed in combination with a light source or ultrasound therapy to determine an increase or decrease in blood flow to a tumor region in response to said therapy. The individual transducer electrodes, as described in FIGS. 7A-7C or FIG. 8, could allow for Continuous Wave Doppler monitoring. The single electrode pattern 224 shown in FIG. 5 could be subdivided into individual rows, allowing the ultrasound beam to be electronically steered (cf. Kremkau, Chapters 3 and 4).

With respect to the alternative design depicted in FIGS. 9A and 9B, this approach would be applicable as a retrofit to an existing transducer soundhead. Ultrasound coupling gel would be used between the faceplate 102 and the opto-acoustic material 420. The design of the material 420 is at the choice of the designer, and may be comprised of a series of internal optical reflectors, each sufficiently thin so as to be nominally transparent to the ultrasound beam. For example, gold foil in thicknesses of less than 50 micron, cast into a clear resin in a conical fashion would reflect much of the light from sources 300, while causing minimal effect on the ultrasound beam. While this approach would not have the advantages of the design described above, including complete overlap of the ultrasound and light beams, uniformity of the ultrasound and light beams, capability of shaping the transducer surface, etc., it does allow for retrofitting existing soundheads, which can afford a commercial advantage.

With respect to the alternative design depicted in FIGS. 10, 11A and 11B, this approach would be a lower cost alternative to the piezocomposite design, and would, depending upon the number of rings used, allow more light to pass through. It would, however, generally produce an ultrasound field with lower intensity and with less uniformity that the approaches described earlier. Thus the designer has a number of choices to balance with these various design approaches. The more that the surface area of the transducer is taken up with ultrasound elements, the lower the light output, but the higher and more uniform the ultrasound. The 1-3 composite approach can, at best, have only 75 percent of the possible light output of an equivalent area filled only with lighting elements; the 2-2 approach is even more limited, with around 50 percent (depending upon the ratio of the transducer element width to the element-to-element spacing). It is clear to those skilled in the art that these ratios can be changed to adjust the ultrasound output and the light output as the demands of the clinical situation dictate. The invention disclosed herein affords the designer great flexibility in meeting the clinical requirements for simultaneous, co-located ultrasound and phototherapy treatment.

As described in detail above, it is advantageous to co-locate the simultaneous treatment of ultrasound and light onto the same area of the tissue to be treated as a result of complementary effects achieved by both types of applied energy together. It is often necessary in such applications, like medical or cosmetic applications, to reach a threshold level of ultrasonic pressure or light amplitude in order to effect an adequate treatment. Such ultrasound pressures may be greater than 100 kPA, such as approximately 100 kPa, 200 kPa, or 500 kPa, for example, and such light intensities may be varied, with test examples of approximately 30 mW/cm², 50 mW/cm², or 100 mW/cm², for example. However, if the ultrasound energy is delivered in a continuous manner (i.e. with a continuous, uninterrupted amplitude, as defined by international standards and as known in the engineering arts), then the total power delivered may be sufficiently high to cause a temperature rise and potentially cause tissue damage. Therefore, it may be advantageous to deliver ultrasound energy in a pulsed mode, in which a first higher amplitude burst of ultrasound energy of a finite duration is followed by a second lower amplitude (or zero amplitude) burst of energy of an equal, smaller, or longer duration. This pattern may be repeated for the full extent of the treatment period. Advantageously, by pulsing ultrasound energy, it is possible to deliver instances of higher pressure amplitude while maintaining the same overall power level as a continuous application of lower pressure. In various embodiments, light energy may be pulsed in a similar manner. In this regard, the pulsing of energy enables more energy-efficient use of the treatment device while maintaining the effectiveness of the treatment method applied to the tissue. A schematic comparison of these continuous and pulsed treatment methods and a description of the functional benefits thereof are provided in detail in the following paragraphs.

With reference to FIGS. 12 through 15, several embodiments of continuous and pulsed energy application are shown graphically. It will be understood that continuous or pulsed energy application, or combinations thereof, may be employed in connection with the delivery of ultrasound and light energies by any one of the photo-acoustic treatment devices described herein.

As shown in the graph of FIG. 12, ultrasound and/or light may be delivered as a continuous wave of energy, referred to as Continuous Wave (CW) or Continuous Mode treatment. With Continuous Wave treatment, there is no temporal interruption of the energy deposition throughout the duration of the treatment. The “Amplitude” denoted by the vertical axis of the graph in FIG. 12 may refer to the amplitude of an ultrasonic pressure wave or the amplitude of light, for example. The horizontal axis represents progression of time during a period that an energy emitting element (e.g., an ultrasound transducer or a light source) is energized to emit energy for treatment, and during which period the emitted wave of energy may continuously rise and fall in a sinusoidal manner, as indicated by the magnified portion of the graphed wave. As described above, Continuous Wave application of ultrasound energy may undesirably prove destructive to host tissue at some high energy or power levels, and thus, pulsed application of energy is preferred in some applications, as described below.

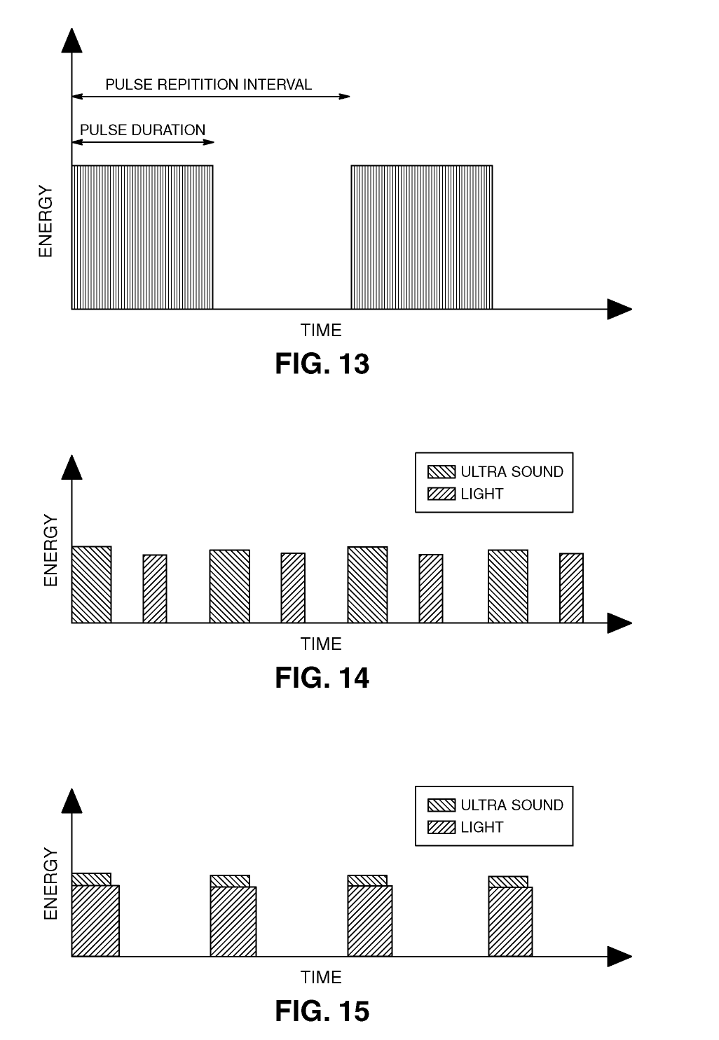

In another embodiment of treatment, ultrasound energy and/or light energy directed at a tissue may be delivered in a pulsed mode for a full or partial portion of a treatment period. With reference to FIG. 13, a pulsed waveform of an energy (e.g., light or ultrasound) is shown in simplified form, illustrating several discrete pulses that are periodic and separated in the illustrated view. It will be understood that the pulsed waveform may be of a sinusoidal or similar shape, comprising both positive and negative energy values (e.g. pressure of a sound waveform, or amplitude of a light waveform). As shown, the time period during which the energy is turned “on” (i.e., emitted) is referred to as a Pulse Duration (PD), or Pulse Length. The time period over which the pulse repeats is referred to as the Pulse Repetition Interval (PRI). The reciprocal of the Pulse Repetition Interval is referred to as the Pulse Repetition Frequency (PRF). The Duty Cycle (DC), or Duty Factor (DF), is a ratio of the Pulse Duration to the Pulse Repetition Interval (PD/PRI), and indicates the percentage of a given time period that the energy is on. As such, a Duty Cycle of 100%, in which the Pulse Duration is equal to the Pulse Repetition Interval, indicates Continuous Wave operation. A Duty Cycle of less than 100% refers to pulsed operation of periodic (or non-periodic) on and off cycles or pulses of energy. The Duty Cycle also represents the percentage of the maximum possible energy delivery for a given wave amplitude.

When delivering both ultrasound and light to a target, whether biological (e.g. tissue) or non-biological (e.g. a ship hull, medical catheter, or other structure coated with biological material), the two energies may be spatially coincident (e.g., “co-located”), meaning that both energies may be directed to the same location in space. In other words, the beams of energy may overlap spatially. As described above, due to the nature of the ultrasound energy and light energy delivery processes and the potential positive and/or negative reactions of biological materials to these energies impinging upon biological materials, it may be advantageous to deliver the combined energies (e.g., one or both of the ultrasound energy and the light energy) in a pulsed mode rather than a continuous mode. Further, it may be advantageous to deliver the pulsed ultrasound and/or light energies with varying degrees of temporal overlap, including full temporal overlap, partial temporal overlap, or no temporal overlap, for example.

There are several reasons why one skilled in the art would choose to deliver the ultrasound and light energies in a pulsed manner rather than a continuous manner. As described above, it may be necessary to generate a certain level of pressure or light energy in order to effect an adequate treatment in a given application. The attenuation of both sound and light through tissue generally limits the depth of penetration of each type of energy. By increasing the amplitude of an energy, the energy can reach a greater depth. Increased amplitude in Continuous Wave application may cause unwanted heating and degradation of the energy emitting instrument (e.g., a piezoceramic transducer or LED light source) or unwanted heating and degradation of the targeted tissue, as described above. By pulsing the emitted energy, the average energy level may be maintained at a safe degree while the peak energy may be increased to effect the desired treatment. For example, the temperature rise is essentially directly related to total power applied, so if a similar amplitude energy were applied using pulsing with a 10% Duty Cycle, the temperature increase in tissue being treated would be 10% of the temperature increase that would occur with the same amplitude energy application in a Continuous Wave setting. Reducing the total energy application can also allow blood flow to the tissue to partially or fully compensate for the energy application, thus limiting any temperature increase, while the effectiveness of the treatment remains essentially the same because of the ultrasound/light effects lasting beyond the time period when the energy is actually being applied. To this end, the treatment device may define a threshold amplitude for light energy and ultrasound energy above which degradation of the treatment device or damage to the tissue would occur during a Continuous Wave operation of the treatment device, and the pulses of ultrasound energy and the pulses of light energy are emitted at a maximum amplitude greater than the threshold amplitude without risk of damaging the tissue because of the pulsed operation.

An additional reason for using ultrasound in a pulsed mode relates to the potential problem of standing waves. When ultrasound travels from a first medium to a second medium having a different acoustic impedance than the first medium (e.g., from tissue to bone, water to plastic, or water to steel), some amount of the ultrasound energy may be reflected back towards the ultrasound transmitter. This reflected energy may interact with the transmitted energy to create regions of higher and lower pressure amplitude which are fixed in space, and which are known as standing wave patterns. Standing wave patterns can be especially problematic when treating patients. For example, standing waves may give rise to undesirable excess heat generation within the tissue. Using ultrasound in a pulsed mode reduces the likelihood of such excess heat generation.

FIG. 14 shows an exemplary treatment with pulsed ultrasound and light in which there is no temporal overlap of the two energies, such that the delivery of the two energies is alternating and interleaved. Additionally, as shown, the Pulse Repetition Interval for the discrete pulses of ultrasound energy and the discrete pulses of light energy may be the same, and the two energies may therefore be effectively synchronous. Of course, in alternative embodiments (not shown in the FIGS.) the Pulse Repetition Interval may not be the same for both energies, leading to some overlapped pulses and other non-overlapped pulses, the energies being not synchronous in such an operation.

A beneficial result of delivering the pulsed ultrasound and light energies as discrete pulses with no temporal overlap, such that the energies are alternated and interleaved as shown in FIG. 14, is maintaining a balanced energy load on the energy generation equipment at the treatment device. In this regard, a power supply of the treatment device may be limited in operation to a particular average power consumption. When the emitted energy pulses are overlapped temporally, they create a combined power consumption that is higher than an individual power consumption corresponding to either one of the energies, and which combined power consumption might undesirably extend beyond the operational limits of a particular power supply of the treatment device. This requirement for a more robust power supply is not a concern when using the non-overlapping treatment method set forth in FIG. 14. Thus, the test arrangement and method indicated in FIG. 14 provides high equipment cost efficiency and treatment effectiveness.

FIG. 15 shows another exemplary treatment with pulsed ultrasound and light in which the two energies fully overlap temporally during a portion of the overall Pulse Repetition Interval, each of the energies having the same Pulse Repetition Rate. As alluded to above, the Pulse Duration of the light energy may be greater than or less than (and in other embodiments, equal to) the Pulse Duration of the ultrasound energy, so as to deliver the light energy with a Duty Cycle greater than or less than (and in other embodiments, equal to) a Duty Cycle of the ultrasound energy. Additionally, where the ultrasound and light energies fully overlap temporally, they may be synchronized such that the respective pulse of each energy starts at the same time. This is the arrangement of the energy plot shown in FIG. 15, for example. When using fully overlapping energy such as shown in this embodiment, there is no reduction of the ultrasound effect on tissue (e.g., reduction which would be caused by time passing after the ultrasound energy stops being applied) while the light is being applied, in view of the two energies being applied simultaneously in time. Of course, a higher power source of energy may be required when overlap of the two energy applications is required, particularly as shown in FIG. 15. To this end, the maximum power input being provided by a power source on the treatment device needs to be higher in this operation than in FIG. 14 (put another way, the power source for FIG. 14 can be inexpensive and less robust, even though such a power source would likely be insufficient to provide simultaneous overlapping pulses of ultrasound energy and light energy as shown in FIG. 15).

Additionally, with respect to a light source (e.g., an LED) of a photo-acoustic treatment device, a maximum amplitude of the light may be set by controlling the maximum current through the light source. The average light output may be set by adjusting the Duty Factor. Any suitable controller may be used for performing these functions, such as a CD400-DMX controller made available by Digital Lighting Systems, for example. The controller may operate with a fixed Pulse Repetition Frequency of 100 Hz (PRI of 10 milliseconds), and a Duty Factor which may be adjusted digitally over a range of 0 to 255, which corresponds to a Duty Factor of 0% to 100%, for example. It will be understood that the controller used with the treatment device may control the maximum amplitude, the Pulse Repetition Frequency, and the Duty Factor for both the light sources and the ultrasound transducer. To this end, the controller determines the specific operational parameters used during a treatment method using pulsed application of energies, regardless of the specific overlap or non-overlap nature of the pulsed energy applications.

In one alternative embodiment, and in the interest of mitigating financial burdens on a user, it may be desirable to drive each of the ultrasound and light energy sources with independent power sources, such that there is no synchronization between the two energy sources. For example, the ultrasound source may have a higher or lower Pulse Repetition Frequency than that of the light source, in which case there would potentially be no synchronization between the two energy sources. For example, if the two sources have Pulse Repetitions frequencies that are not related by an integer multiple, then there would be no synchronization between the two series of pulses. This arrangement would result in having some time periods of temporal overlap of ultrasound and light and other time periods without temporal overlap of ultrasound and light. Depending upon the exact Duty Factor of each energy, the percentage of overlap of the light and ultrasound energies could vary from near zero to near 100%, and could change as a function of time, for example. In this regard, if more periods of overlap are desired during the treatment time, the Pulse Repetition Frequency and Duty Factor for the light and ultrasound sources can be set accordingly.

Although the description of the discrete pulses of ultrasound and light energy above have referred to circumstances where the pulse duration and/or pulse repetition interval remains similar during a treatment period, it is also possible to have the discrete pulses define pulse durations and pulse repetition intervals that are modulated and therefore not equivalent over the course of a treatment. Even in such modulated pulse arrangements, an effective Duty Factor still applies based on the percentage of time that the ultrasound transducer (for example) is active compared to the overall treatment time period. In this regard, the discrete pulses of energy used with these embodiments of the disclosed invention may define a periodic pattern or may be a modulated non-periodic pattern, without departing from the scope of this disclosure. In one particular example of a non-periodic pattern, the pulses of ultrasound energy may be modulated based on control signals from a digital music player such that the ultrasound produces an approximation of a desired song or music on the digital music player. The ultrasound would consequently be controlled similarly to a speaker output typically used with the digital music player. If the treatment device were being used to treat acne on a teenaged patient, in such an arrangement, the patient could advantageously “hear” the music that he desires being played through the face by the discrete modulated pulses of the ultrasound transducer. Accordingly, the pulsed delivery of energy in the treatment device could potentially provide positive treatment experiences and ancillary benefits for a patient as well as the other technical benefits and advantages described herein.

Through experimentation, it has been discovered that a range of Pulse Durations and Pulse Repetition Intervals for the discrete pulses of ultrasound and light may be effective for killing bacteria, which is one exemplary type of tissue treatment that may be performed with the treatment device and method of this disclosure. For example, Pulse Repetition Intervals of 2 milliseconds to 1000 milliseconds; Pulse Durations of at least 1 millisecond; with ultrasound Duty Factors of 1% to 50% and light Duty Factors of 2% to 100% are most effective for treatment of tissue and various biofilms, regardless of whether the discrete pulses are periodic or modulated. Moreover, it has been found that separate extended exposures (e.g., such as several minutes long) of ultrasound only and then light only, or the reverse, are not as effective for killing bacteria. This result is believed to occur because the ultrasound effect on bacteria, which, for instance, may enhance the susceptibility of the bacteria to treatment by light, is relatively short lived (e.g., only a few seconds). Accordingly, in one embodiment, any spatially coincident ultrasound and light treatment may be applied to the bacteria within less than approximately one second of each another. For example, the ultrasound energy delivery may be repeated at least once every other second. In this manner, a cumulative dose of energies may be delivered to the bacteria, because there is not enough time between pulses for the collective and/or combined bactericidal effect of the energies to diminish significantly.

Within the desired operational parameters provided in the previous paragraph, the Duty Factor of the ultrasound defines or controls the overall bacteria killing or treatment effect once a threshold ultrasound pressure (e.g., above 100 kPa) has been reached. To this end, the Pulse Duration and Pulse Repetition Interval can be modified while maintaining a similar Duty Factor, and this will result in similar treatment effectiveness and results. By contrast, when the Pulse Duration is changed without changing the Pulse Repetition Intervals, again within the desired operational parameter ranges provided above, the Duty Factor changes and the treatment effectiveness also changes. To this end, if more effective treatment or killing of bacteria is required, it is more effective to increase the Duty Factor than the pressure or power applied in the ultrasound. However, desirable treatment results for various applications will be achieved using many different values within the operational parameter ranges above, as evidenced by test results, some of which are described below.

Accordingly, a pulsed mode of energy exposure may be just as effective as a Continuous Wave mode of energy exposure, while also enabling the use of potentially higher peak energy amplitudes for effecting enhanced treatment. In this regard, targeted bacteria (e.g., on a tissue) does not respond quickly enough to sense a difference between continuous excitation and rapidly pulsing excitation (e.g., at 10 times per second or greater), but the various potential problems with Continuous Wave application of energy like overheating or standing waves are not encountered with the rapidly pulsing excitation. Additionally, when treating bacteria and where the mechanism of action of ultrasound is through the force that is applied to the bacteria through acoustic radiation force, energy pulsations may have a greater effect on the bacteria than continuous excitation, which produces a constant force on the bacteria. In that regard, bacteria cells or other types of tissue may adapt to constant forces, and thus the effect provided by a constant force may diminish quickly with time. By pulsing the energies, the bacteria and/or the tissue cannot adapt to the changing conditions, and the advantageous combined effect of the energies may continue with high efficiency throughout the treatment time. This combined effect with pulsed treatment occurs when treating other types of cells and tissue as well.

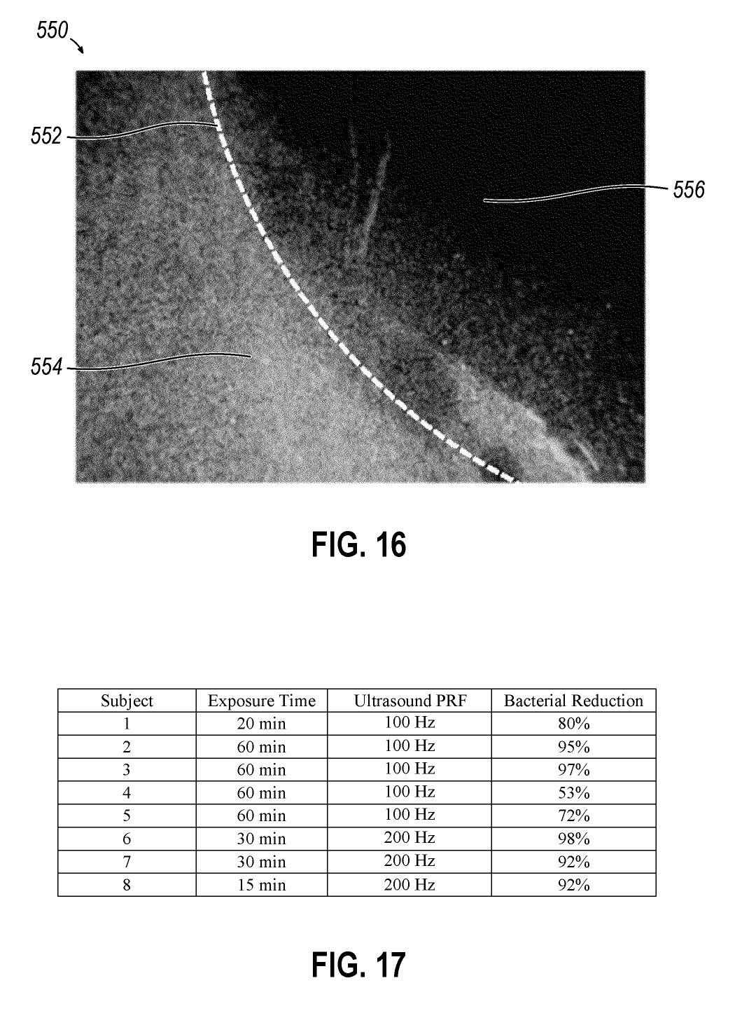

Several experiments proving the effectiveness of combined ultrasound and light treatment as well as pulsing ultrasound and light treatment are now summarized with reference to FIGS. 16 through 18. Beginning with reference to FIG. 16, an acne bacteria biofilm 550, stained with a dye, is shown after having been exposed to two different types of treatment on either side of diagrammatic line 552. In particular, a first region 554 of the biofilm 550 on the left side of the line 552 was exposed to light alone, and its lighter color indicates a predominant presence of living biofilm cells after treatment. A second region 556 of the biofilm 550 on the right side of the line 552 was exposed to combined ultrasound and light (e.g., pulsed ultrasound and light in overlapping and/or non-overlapping time periods), and its darker color indicates a predominant presence of dead biofilm cells after treatment. Accordingly, the experimental results shown in FIG. 16 demonstrate that combined ultrasound and light treatment is more effective than light treatment alone for damaging and/or killing bacterial biofilm cells, which is one typical type of tissue treatment desired with the treatment devices of the present disclosure.

During another exemplary experiment, the results of which are summarized in FIG. 17, subjects were exposed to combined ultrasound and light using a treatment device 612 described in further detail below and held in direct contact with the skin of each subject at an emitting end thereof. In this manner, bacteria residing on the skin at levels beneath the outermost surface of the skin (e.g., at 0.1 to 0.9 mm beneath the outermost surface of the skin, for example) were coupled to the photo-acoustic element of the treatment device in indirect contact established through intervening (e.g., overlying) layers of skin. In that regard, it will be understood that coupling via indirect contact between bacteria and a photo-acoustic treatment device treating the bacteria may be established through one or more layers of overlying bodily tissue (e.g., skin) positioned between the bacteria and an energy emitting surface of the treatment device. Further, as described above and below, coupling via indirect contact between bacteria and a photo-acoustic treatment device may be established through an acoustic coupling medium, such as an acoustic coupling liquid, positioned between the bacteria and an energy emitting surface of the treatment device. Accordingly, contact is always provided between the bacteria and the photo-acoustic treatment device, even when a “direct contact” with the bacteria is rendered impossible by the test setup or by the physical location of bacteria on the body.

The treatment device 612 was controlled to provide an ultrasound Pulse Repetition Frequency of 200 Hz and an ultrasound pulse duration of 275 microseconds, or alternatively, a Pulse Repetition Frequency of 100 Hz and a pulse duration of 550 microseconds. Both combinations produced the same intensity and overall applied power levels. The treatment device was further controlled to provide a light Pulse Repetition Frequency of 100 Hz and a light Duty Cycle of 95%. The subjects were exposed to the combined pulsed ultrasound and light for periods varying between 15 minutes and 60 minutes. Skin swabs were then taken and the collected bacteria was cultured for 7 days in growth medium. The resultant Colony Forming Units of P. acnes bacteria were then counted. On each subject, two regions were sampled: one region which had been treated, and another untreated control region nearby (e.g., an untreated control region at a contralateral position on a subject's face). The aforementioned step provided a measure of the nature variation in bacterial levels for each subject. Differences between the sample taken before the treatment and 24 hours after the treatment were noted, with corrections made based on the untreated control regions.

The above-described operating parameters and corresponding experimental results for each patient are summarized in the table shown in FIG. 17. As shown in the experimental results summary of FIG. 17, the bacterial reduction rates ranged in all but one subject from 72% to 98% with combined, pulsed light and ultrasound energies. The sample plates used for test subject 4 appeared to contain other types of bacteria beyond P. acnes, which negatively affected the numerical results, and likely can be disregarded therefore. Although not shown in the table of FIG. 17, exposure to light alone produced an average bacterial reduction of 25%, and exposure to ultrasound alone produced no measurable bacterial reductions. These results are consistent with those shown in the experiment of FIG. 16, and further indicate that a pulsed, spatially overlapping or “co-located” application of combined light and ultrasound energies provides an effective and substantial treatment for killing bacteria. Similar beneficial effects may be observed in other types of tissue treatments and therapies provided when combining ultrasound and light energy, but these experimental test results provide a sufficient explanation of those benefits regardless of the particular treatment application.

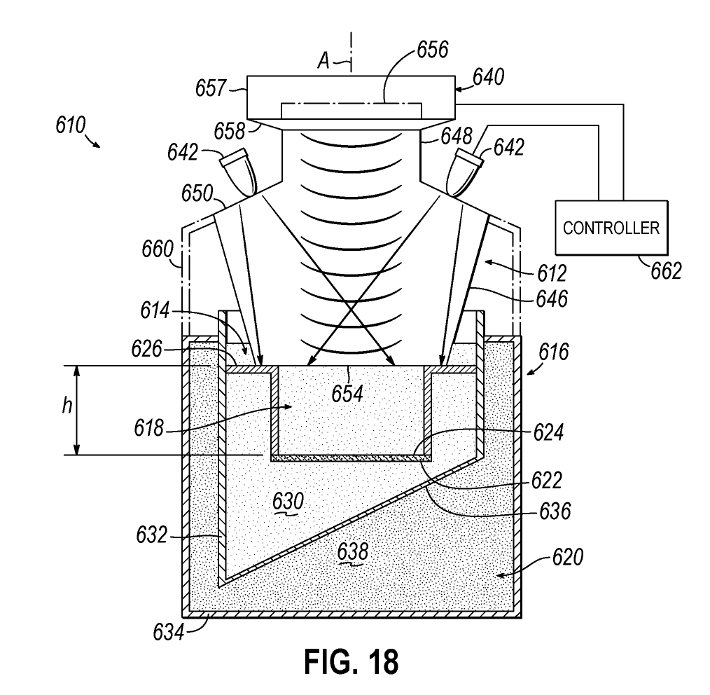

The treatment device 612, which is used to produce the test results of FIG. 17, is shown schematically in cross section in FIG. 18 along with the remainder of an experimental setup 610. This is yet another alternative embodiment of the treatment devices described above, with many similarities regarding the ultrasound transducer and light sources used. The setup 610 may include the treatment device 612, a cell culture insert 614, and a container assembly 616 having a treatment chamber 618 and an absorption chamber 620. The setup 610 is described herein in the context of treating a bacterial biofilm. However, the photo-acoustic treatment device 612 may be also be used for treatment of bacteria in a planktonic state using similar or different operating parameters, as described in greater detail below. The treatment device 612 is also configured to be used on a patient's skin in actual clinical use, not just on test samples held by the cell culture insert 614 as shown.

The cell culture insert 614 may be of any type of structure suitable for growing and harvesting a bacterial biofilm. The insert 614 may include a base surface 622 in the form of a permeable membrane, on which a biofilm 624 may be grown. For example, the base surface 622 may be formed of a thin, permeable layer of polyethylene terephthalate (“PET”) of the kind found on a Millicell® hanging cell culture insert. The insert 614 may further include a flange 626 extending radially for supporting the insert 614 within the treatment chamber 618, as shown in FIG. 18. The flange 626 is also sized to support the leading end of the treatment device 612 as described in further detail below.

The treatment chamber 618 of the container assembly 616 is configured to retain an amount of sterile fluid 630, such as phosphate buffered saline (“PBS”), and may be sized such that the base surface 622 of the cell culture insert 614 may be received therein. The treatment chamber 618 is defined by an outer wall 632, which may be in the form of a hollow cylinder extending axially, as shown. A bottom portion of the outer wall 632 may be angularly formed relative to a bottom wall 634 of the absorption chamber. The bottom portion of the treatment chamber 618 may be sealed with a fluid-impermeable membrane 636 to prevent fluid exchange between the treatment chamber 618 and the absorption chamber 620. The membrane 636 may be in the form of a thin plastic web, for example.

The absorption chamber 620 of the container assembly 616 is configured to retain an amount of a viscous fluid 638, such as castor oil. The viscous fluid 638 may have a viscosity sufficient to absorb ultrasound energy transmitted to the viscous fluid 638, as described below. As shown, the treatment chamber 618 may extend into the absorption chamber 620 such that the angled bottom portion of the treatment chamber 618 is submersed in the viscous fluid 638 and is suspended above the bottom wall 634. The treatment chamber 618 may be coupled to the absorption chamber 620 with any suitable type of fastening or connection.

The photo-acoustic treatment device 612 may include an ultrasound transducer 640 with an emitting surface, one or more light sources 642, and a photo-acoustic element 646 that is arranged between the combined ultrasound transducer 640 and light sources 642 and the cell culture insert 614. In one embodiment, the photo-acoustic element 646 is generally frusto-conical in shape (e.g., the leading end contacting the flange 626 is a truncation of the conical shape), and includes a cylindrical upper extension 648 extending axially opposite the leading end and a shoulder 650 defined at the base of the upper extension 648. The shoulder 650 may be angled relative to the longitudinal axis A of the photo-acoustic element 646 as shown, or alternatively the shoulder 650 may be substantially transverse to the axis A. The photo acoustic element 646 may be a solid structure formed of a translucent, photo-acoustic material configured to transmit light and ultrasound energy simultaneously therethrough and to confine the light and ultrasound energy within its interior with minimal loss and attenuation. For example, the photo-acoustic element 646 may be comprised of thermoplastic or thermosetting polymer, such as an acrylic resin. In the experimental setup 610 shown, the diameter of the circular aperture at the bottom of the photo-acoustic element 646, which is also configured to be in contact with the subject's skin when not used in the test arrangement setup 610, is 29 millimeters. Advantageously, in the context of experimental setup 610, this emitting end 654 is sufficient in size to span across substantially a full diameter of base surface 622 containing biofilm 624, thereby enabling the treatment device 612 to provide treatment to a full portion of biofilm 624

The light sources 642 may be in the form of at least one light emitting diode (“LED”), for example UVSTZ-405015 LEDs made available by Bivar, Inc. of Irvine, Calif. While only two light sources 642 are shown, any suitable number of light sources may be used. For example, eight or more light sources may be used, or a single light source may be used. As shown, the light sources 642 may be arranged circumferentially about the upper extension 648 and may be supported by a structure (not shown) that may be mounted to the photo-acoustic element 646 coaxially such that the light sources encircle the upper extension 648 and are positioned adjacent to the shoulder 650. The light sources 642 may be positioned angularly relative to the axis A of the photo-acoustic element 646, and are operable to direct light downwardly through the element 646 such that the light transits through the element 646 and is emitted through the emitting end 654 (also referred to as leading end) of the element 646, as indicated by the elongate arrows.

The ultrasound transducer 640 of the illustrated embodiment may include a transducer element 656 located within a larger housing 657 and a faceplate 658 coupled directly or indirectly to and spanning a width of the transducer element 656. The transducer element 656 may include a piezoelectric ceramic element in the form of a disk, and the housing 657 and faceplate 658 may be formed of any suitable metal such as aluminum, for example. It will be appreciated that the faceplate 658 may also be formed of other ultrasound transmissive materials such as plastics, and also that the faceplate 658 may be omitted in other embodiments in which the transducer element 656 is coupled or bonded directly to the photo-acoustic element 646. Likewise, the housing 657 may be omitted in other embodiments, such as where the transducer element 656 is mounted on a plate, for example, which could then transfer the ultrasound energy into the photo-acoustic element 646. Alternatively, the ultrasound transducer 640 may be constructed as generally shown and described in U.S. Pat. No. 8,206,326, for example. In particular, the transducer element 656 may include a plurality of smaller individual transducer elements (not shown herein) embedded in or otherwise surrounded by a polymer matrix material so as to form a piezocomposite structure. Generally, the transducer element 656 may emit ultrasonic waves when alternating electrical current is applied to its electrodes. While in the configuration as shown, the ultrasonic waves comprise longitudinal waves, other configurations may generate and transmit to the tissue other types of ultrasonic waves, such as transverse waves, torsional waves, shear waves, surface waves, Raleigh waves, or Lamb waves, for example. As shown, the transducer 640 may be mounted coaxially with the photo-acoustic element 646 such that the faceplate 658 abuts a top surface of the upper extension 648. In this manner, the faceplate 658 may be acoustically coupled to the photo-acoustic element 646 for transmission of ultrasound energy downwardly through the element 646 and outwardly through the emitting end 654 thereof, as shown generally by arcuate lines.

As described above, the emitting end 654 of the photo-acoustic element 646 is supported on the flange 626. Optionally, a support structure 660 of any suitable size or shape may also be used to properly align the treatment device 612 relative to the cell culture insert 614. Whether one or both of these positioning elements is used with the treatment device 612, the device is accurately positioned at a relative height “h” above the biofilm or sample to be treated, and it will be appreciated that this height can be modified by changing the relative dimensions of some of the elements described in accordance with this embodiment. It will also be understood that when the treatment device 612 is used on a patient's tissue rather than in this test arrangement 610, the end of the treatment device 612 at photo-acoustic element 646 is brought directly into contact with the tissue during normal operation (a coupling gel may optionally be present as well).

The ultrasound transducer 640 of treatment device 612 may be controlled to generate ultrasound energy using a function generator (not shown) and a power amplifier (not shown). The function generator and power amplifier may be of any suitable types, for example an Agilent 33220A function generator made available by Agilent Technologies of Santa Clara, Calif., and an ENI 325LA power amplifier made available by Electronics & Innovation of Rochester, N.Y. Acoustic energy output by the ultrasound transducer 640 may be measured using any suitable sensor, such as a Reson TC4038 hydrophone made available by Teledyne Reson of Slangerup, Denmark and a model 804 bilaminar membrane hydrophone made available by Acertara Acoustic Laboratories of Longmount, Colo., for example.

The plurality of light sources 642 of treatment device 612 may be digitally controlled to create light energy using any suitable computer, a USB controlled interface module, and a four-channel pulse-width-modulation (“PWM”) light control unit (not shown), for example. The interface module may be a VM116 model made available by Velleman, Inc. of Fort Worth, Tex., and the light control unit may be a CD45 model made available by Digital Lighting Systems of Miami, Fla., for example. Light energy output by the light sources 642 may be measured using any suitable sensor, such as an ILT400 radiometer and a SEL033/W detector made available by International Light Technologies of Peabody, Mass., for example. In one embodiment, the ultrasound transducer 640 and light sources 642 may be controlled by a common controller 662, for example.

As noted above, the controller 662 is configured to operate the ultrasound transducer 640 and the light sources 642 in a pulsed manner rather than a continuous manner. In this manner, the controller 662 operates the treatment device 612 so as to create a certain high level of pressure or light energy to be effective at certain tissue depths while avoiding unwanted heating and degradation of the treatment device 612 that could be caused when operating continuously at the high level or pressure or light energy. Furthermore, where the pulsed ultrasound and light energies are interleaved with one another and not overlapping, a balanced and lower maximum energy load is necessary from the power source(s) of the treatment device 612. Thus, as in the acne biofilm treatment example provided above, the treatment device 612 enables combined energy therapy which is beneficial and advantageous over known designs and treatment methods.

While preferred treatment devices and methods have been described in detail, various modifications, alterations, and changes may be made without departing from the spirit and scope of the present invention as defined in the appended claims.

What is claimed is:

1. A method for treating tissue, the method comprising:
contacting a portion of a treatment device with the tissue;
generating ultrasound with the treatment device;
generating light with the treatment device;
transmitting the ultrasound and the light from the treatment device into the tissue during contact with the tissue, such that the ultrasound and the light are at least partially co-located on the tissue; and
treating the tissue with both the ultrasound and the light generated by the treatment device to provide a therapeutic effect to the tissue where the ultrasound and the light are co-located,
wherein at least one of the ultrasound and the light is generated in discrete pulses.

2. The method of claim 1, wherein generating ultrasound with the treatment device further comprises:
generating discrete pulses of ultrasound energy for application on the tissue, the discrete pulses of ultrasound energy defining a first pulse duration and a first pulse repetition interval.

3. The method of claim 2, wherein the first pulse duration and the first pulse repetition interval are modulated so as to be varied over a treatment time.

4. The method of claim 2, wherein generating light with the treatment device further comprises:
generating discrete pulses of light energy for application on the tissue, the discrete pulses of light energy defining a second pulse duration and a second pulse repetition interval.

5. The method of claim 4, wherein the first and second pulse repetition intervals are equal such that the discrete pulses of ultrasound energy and the discrete pulses of light energy are synchronous.

6. The method of claim 5, wherein the discrete pulses of ultrasound energy and the discrete pulses of light energy are alternating and non-overlapping in time during treatment of the tissue with both the ultrasound and the light.

7. The method of claim 5, wherein the discrete pulses of ultrasound energy and the discrete pulses of light energy are at least partially overlapping in time during treatment of the tissue with both the ultrasound and the light.

8. The method of claim 4, wherein the first and second pulse repetition intervals are not equal such that the discrete pulses of ultrasound energy and the discrete pulses of light energy are not synchronous.

9. The method of claim 1, wherein the light is generated with at least one light emitting diode of the treatment device, and the ultrasound is generated with a piezoelectric ceramic element.

10. The method of claim 1, wherein treating the tissue with both the ultrasound and the light further comprises:
treating acne bacteria by applying the ultrasound and light via indirect contact between the treatment device and the acne bacteria, thereby resulting in the therapeutic effect of killing the acne bacteria.

11. The method of claim 1, wherein transmitting the ultrasound and the light further comprises:
transmitting the ultrasound and the light from the portion of the treatment device in contact with the tissue.

12. The method of claim 1, wherein the ultrasound generated by the treatment device is therapeutic ultrasound, and the light generated by the treatment device is therapeutic light.

13. The method of claim 1, wherein the tissue includes a wound, and the therapeutic effect is effective to treat the wound.

14. The method of claim 1, wherein the tissue comprises a non-melanoma skin cancer that receives co-located treatment with the ultrasound and the light.

15. The method of claim 1, wherein the tissue comprises acne that receives co-located treatment with the ultrasound and the light.

16. The method of claim 1, wherein the tissue is photo-rejuvenated by receiving co-located treatment with the ultrasound and the light.

17. The method of claim 1, wherein a permeability of the tissue is changed.

18. A treatment device for treating a tissue, the treatment device comprising:
an ultrasound transducer operable to produce ultrasound;
a light source operable to product light;
a photo acoustic element operatively coupled to the ultrasound transducer and positioned relative to the light source such that the ultrasound and light are transmitted through the photo acoustic element for transmission to the tissue when a portion of the treatment device is contacted with the tissue, the photo acoustic element enabling the ultrasound and the light to be at least partially co-located at the tissue; and
a controller operatively coupled to the ultrasound transducer and the light source, the controller operating at least one of the ultrasound transducer and the light source to produce discrete pulses of ultrasound energy and/or discrete pulses of light energy, the controller functioning to operate the ultrasound transducer and the light source to treat the tissue with both the ultrasound and the light and thereby provide a therapeutic effect to the tissue where the ultrasound and the light are co-located.

19. The treatment device of claim 18, the controller operating the ultrasound transducer to generate discrete pulses of ultrasound energy defining a first pulse duration and a first pulse repetition interval and also operating the light source to generate discrete pulses of light energy defining a second pulse duration and a second pulse repetition interval, with the first and second pulse repetition intervals being equal such that the discrete pulses of ultrasound energy and the discrete pulses of light energy are synchronous.

20. The treatment device of claim 19, the controller alternating the discrete pulses of ultrasound energy and the discrete pulses of light energy so as to be non-overlapping in time during treatment of the tissue with both the ultrasound and the light.

21. The treatment device of claim 19, the controller actuating generation of the discrete pulses of ultrasound energy and the discrete pulses of light energy so as to be at least partially overlapping in time during treatment of the tissue with both the ultrasound and the light.

22. The treatment device of claim 18, the controller operating the ultrasound transducer to generate discrete pulses of ultrasound energy defining a first pulse duration and a first pulse repetition interval and also operating the light source to generate discrete pulses of light energy defining a second pulse duration and a second pulse repetition interval, with the first and second pulse repetition intervals being unequal such that the discrete pulses of ultrasound energy and the discrete pulses of light energy are not synchronous.

23. The treatment device of claim 22, further comprising:
a first power source operatively coupled to the ultrasound transducer; and
a second power source operatively coupled to the light source, the first and second power sources being independent so that the controller can operate the discrete pulses of ultrasound energy and the discrete pulses of light energy in the not synchronous manner.

24. The treatment device of claim 18, wherein the photo acoustic element is formed from a plastic or polymer material that is translucent to the light.

25. The treatment device of claim 18, wherein the ultrasound transducer is a piezocomposite transducer element including opposite faces, one of which faces the light source and the other of which contacts the photo acoustic element, such that the light is transmitted through the opposite faces of the piezocomposite transducer element and the photo acoustic element to reach the tissue at the same location as the ultrasound being transmitted from the piezocomposite transducer element.

26. The treatment device of claim 18, wherein the photo acoustic element includes opposing faces with a periphery extending between the opposing faces, one of the opposing faces being in contact with the ultrasound transducer to enable transmission of the ultrasound into the photo acoustic element, and the light source is directed at the periphery of the photo acoustic element, such that the light is transmitted and distributed into the tissue by the photo acoustic element at the same location as the ultrasound.

27. The treatment device of claim 18, wherein the light source includes at least one light emitting diode that generates therapeutic light, and the ultrasound transducer includes a piezoelectric ceramic element that generates therapeutic ultrasound.
